(19) United States
(12) Patent Application Publication
(10) Pub. No.: US 2023/0094020 A1
(43) Pub. Date: Mar. 30, 2023

(54) FACEMASK ADAPTED TO COUPLE WITH AN AIRWAY DEVICE GAS LINE OPENING

(71) Applicant: Eric Franklin HOLT, Washington Grove, MD (US)

(72) Inventor: Eric Franklin HOLT, Washington Grove, MD (US)

(21) Appl. No.: 17/951,189

(22) Filed: Sep. 23, 2022

Related U.S. Application Data
(60) Provisional application No. 63/247,826, filed on Sep. 24, 2021.

(51) Int. Cl. A41D 13/11; A61M 16/06

(52) U.S. Cl. CPC A41D 13/1107; A61M 16/0683

(57) ABSTRACT

The present invention relates to a single or double sided facemask for children to wear either as a stand-alone facemask or for use in conjunction with an anesthesia or airway mask. The double sided face mask has a front surface representing a face and a back surface representing a face. The mask has an aperture in about the nose or mouth area which is adapted to couple with an airway device gas line opening. The face mask is disposable and is easily stored and used. The face mask is designed to couple with a conventional airway or anesthesia mask. Use of the facemask during a medical procedure can place a child at ease and increases acceptance of airway or anesthesia masks.

The present invention relates to a double sided facemask for children to wear either as a stand-alone facemask or for use in conjunction with an anesthesia or airway mask. The double sided face mask has a front surface representing a face and a back surface representing a face. The mask has an aperture in about the nose or mouth area which is adapted to couple with an airway device gas line opening. The double sided face mask is disposable and is easily stored and used. The double sided mask is designed to couple with a conventional airway or anesthesia mask. Use of the mask during a medical procedure can place a child at ease and increases acceptance of airway or anesthesia masks.

Another aspect of the present invention relates to a single sided facemask for children to wear either as a stand-alone facemask or for use in conjunction with an anesthesia or airway mask. The single sided face mask has a front surface representing a face. The mask has an aperture in about the nose or mouth area which is adapted to couple with an airway device gas line opening. The flat single sided face mask is disposable and when not in use is easily shipped and stored flat. The single sided mask is designed to couple with a conventional airway or anesthesia mask. Use of the mask during a medical procedure can place a child at ease and increases acceptance of airway or anesthesia masks.

### OBJECT

A trip to the doctor's office or hospital can be stressful for a child and/or their parents. An airway or anesthesia mask attached to a child's face can be particularly stressful. An airway or anesthesia mask placed over an unsuspecting or noncompliant child's mouth and/or nose, could result in short/long-term emotional distress and real-time negative physiologic responses (i.e., increased heart rate, oxygen consumption, shortness of breath, bronchoconstriction, etc.), thus degrading medical outcome and the child's experience. The object of the invention is to provide a double sided facemask that the child can view at all times. Another aspect of the invention is to provide a single sided facemask. The facemask can be easily and quickly removed, replaced, and reapplied at any time during the medical procedure, therapy or visit. Quick removal of this facemask allows unfettered viewing and manipulation of the anesthesia or airway mask by the medical provider. Upon further study of the specification and appended claims, further objects and advantages of this invention will become apparent to those skilled in the art.

### BACKGROUND OF THE INVENTION

The purpose of an airway device such as an Airway and Anesthesia Mask is to support oxygenation and ventilation. Anesthesia masks are most often constructed of clear material to provide the medical provider an unobstructed view of the patient's mouth/nose for several important reasons. Fogging of an airway mask indicates a patient's airway and the exchange of gases, such as oxygen, carbon dioxide, volatile anesthetic gases, or aerosolized medications. Visualization and inspection of the anesthesia mask on the face by the medical provider can ensure proper anesthesia mask seal in order to deliver the oxygen or agents to the patient. Escaping gas from an improper seal of the mask with the patient's face can be identified visually and audibly allowing corrective measures to be taken in terms of adjusting the fit, size of the airway mask, or additional manual pressure to secure the airway mask. In this way, by monitoring the patient's respiratory effort (chest rise and fall along with nose and mouth visualization), mask placement and proper seal, fogging of the anesthesia mask, and other audible and visual cues, the medical provider can ensure the anesthesia mask is used as intended.

Anything that inhibits the medical provider visualizing respiratory effort or situational awareness (quality of the seal, secretions, emesis, cyanosis of the lips, etc.) is a liability. Prior art masks which embed images on or into the airway mask itself impede visualization. See, for example, U.S. D862,685 and US 2009/0250064

Anything that inhibits the medical provider performing manual adjustments for proper seal of the anesthesia mask impedes optimal care. Prior art with attachments (such as ornamental elephant tusks e.g., US 2009/0250064)) embedded on or into the anesthesia mask impedes proper mask-face seal attempts.

### SUMMARY

The present invention relates to a simple facemask (e.g., a Halloween type mask), that is printed on one or both sides and is adapted to fit over an anesthesia mask. The mask is quicker and easier for a child to process and accept and for medical providers to place and remove from the child.

The invention, in one embodiment, would have images on both sides of the facemask so as to be visible to the child at all times, not just prior to wearing the facemask, enforcing the notion that the child is wearing a mask which happens to maybe contain an anesthesia mask, not the other way around. The facemask straps, if included, can also act to gently secure the anesthesia mask in place without the need for the child or medical provider to hold the mask. The invention, in another embodiment, would have images on one side of the facemask so as to be visible to the child just prior to wearing the facemask, enforcing the notion that the child is wearing a mask which happens to maybe contain an anesthesia mask, not the other way around. The facemask straps, if included, can also act to gently secure the anesthesia mask in place without the need for the child or medical provider to hold the mask. The masks can be stored and shipped flat when not in use.

The invention can be used as a stand-alone facemask before, during, or after the hospital stay. A facemask (or a choice of facemasks) can be easily mailed to the child's address (possibly along with pre-procedure instructions, or a welcome message for the parents) to introduce the patient to the mask prior to the visit. In certain embodiments, the facemask can also be printed with simple lines representing a face that a child can color in with markers, crayons or paint. Once at the medical facility, the facemask can be given to the child or swapped out for an identical or similar facemask (to decrease the risk of home-borne pathogens), enforcing child ownership of the process. Once worn by the patient, the facemask can be easily and quickly removed, replaced, and reapplied at any time during the medical procedure, therapy or visit. Quick removal of this facemask allows unfettered viewing and manipulation of the anesthesia or airway mask by the medical provider. The facemask is easily stored, distributed, and disposed of, all important characteristics for any item used in the medical environment.

The fogging is best visualized with the removal of the single or double-sided face mask, which can be done easily while keeping the oxygen facemask on the child's face. Since the face mask its placed over the oxygen mask and held around the head with an elastic band or similar item it can be moved up and down to chin, up and down to the forehead, or gently taken off totally and then, when the child is waking up, gently place back over the oxygen mask without ever breaking the oxygen mask-to-face seal. Quick disconnects at the facemask-tubing connection make moving, removing, or replacing the mask on the child's face pre- or post-procedure possible without difficulty.

Mask distance from the child's face will be variable and may be on an angle and not directly parallel to the face depending on the shape/contour of the face.

### BRIEF DESCRIPTION OF THE DRAWINGS

Various features and attendant advantages of the present invention will be more fully appreciated as the same becomes better understood when considered in conjunction with the accompanying drawings, in which like reference characters designate the same or similar parts throughout the several views, and wherein:

FIG. 1 depicts a layered view of a double sided facemask (1) of the present invention, a typical anesthesia mask (2) and a patient head.

FIG. 2a depicts the front side of a double sided facemask (1) of the present invention.

FIG. 2b depicts the back side (60) of a double sided facemask (1) of the present invention.

FIG. 3 depicts the front side of a double sided facemask (1) of the present invention.

FIG. 4 depicts the front side of a double sided facemask (1) of the present invention.

FIG. 5 depicts a layered view of a double sided facemask (1) of the present invention, a typical anesthesia mask (2) and a patient head.

FIG. 6 depicts the back side (61) of a single sided facemask of the present invention.

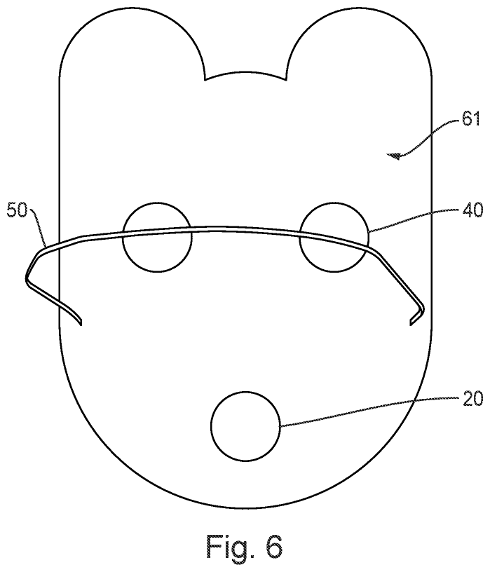

### DETAINED DESCRIPTION OF THE INVENTION

The term “double sided” means that an image representing a face is printed on the mask on both sides of the mask. The same image can be printed on both the front and back surfaces or the front and back surfaces may have different images.

The term “single sided” means that an image representing a face is printed on the mask on only one side of the mask.

Preferably the single side or double side mask is made of a stiff paper. Suitable stiff paper includes for example Cover paper, Tag paper or index paper. Cover paper (typically 60 to 120 pounds) is also commonly referred to as cardstock. Cover paper is thick and stiff (although thickness can vary substantially). It is frequently used for business cards, door hangers, menus, invitations, postcards, rack cards, report covers, self-mailers, sketching, and so on. Index paper (typically 90, 110, and 140 pounds) is stiff paper that is most commonly used for index cards. It's also frequently used for postcards, tabs and dividers, manila folders, and sketchbooks. Tag paper (typically 100 to 200 pounds) is highly durable and fairly stiff. Tag paper is often used for retail signage, price tags, table tents, file folders, door hangers, direct mail postcards, menus, posters, and time cards.

Preferably the single side or double side mask is made of a stiff paper and is essentially flat (i.e., not three dimensional).

A metric measurement (sometimes called grammage) is the actual weight of one sheet of media cut 1×1 meter square. Because the same size sheet is always being weighed, GSM (grams per square meter) provides a more uniform scale to judge a paper's weight and thickness. Thus, paper with a GSM of 140 or above is preferred. Paper with a GSM of 160 or above is most preferred. Paper thickness can be measure using a caliper. Specialty media is often specified in Points or 1/1000 of an inch. For example, media that measures 0.012 inches thick would be marked as 12-point media. In general, paper with a thickness of 0.008 inches thick or above is preferred. Thus, media which is 8-point or above is preferred.

In general, any material that is stiff enough to resist bending or folding under its own weight once coupled to the airway device is preferred.

Most preferably, the double side mask is coated with an anti-infective coating and/or an easy to clean glossy coating. One such suitable paper is ZnO nanoparticles coated paper which is discussed in Ghule et al., Green Chem., 2006, 8, 1034-1041. Other suitable papers include Ag nanoparticle coated paper which is discussed in Kandirmaz et al., in the journal Nordic Pulp & Paper Research Journal; vol. 34, no. 4, 2019, pp. 507-515 or antibacterial paper fabricated by coating acetylated cellulose and cinnamaldehyde for food packaging which is discussed in Zhang et al.; Cellulose volume 28, pages 4371-4384 (2021).

The double sided mask of the present invention may also be made of EVA foam. Preferably, the foam is from about 1-10 mm thick. The mask may also be made of molded plastic such as a traditional Halloween plastic face mask. Typical molded plastic face masks are made of thin PVC or HDPE. Preferably, plastic or foam masks of the present invention are embedded with an anti-microbial agent or are coated with an anti-microbial material. Numerous anti-microbial or anti-viral plastics and polymers are commercially available from, for example, MICROBAN® or BioCote®.

The term “face” as used herein refers to a character with at least on eye and at least one mouth (or beak). Example, characters include animals such as birds, dogs, cats, lions, elephants, pigs, monkeys, foxes, giraffes, pandas, hippos, cows or owls. Cartoon characters are also contemplated. Example, cartoon characters include SPIDERMAN, IRON MAN, SUPERMAN, BATMAN, BUGS BUNNY, MICKY MOUSE, MINI MOUSE, POPEYE, CHARLIE BROWN, HOMER SIMPSON, BART SIMPSON, MARGE SIMPSON, LISA SIMPSON, SNOOPY, FRED FLINTSTONE, PATRICK STAR, BETTY BOOP, WINNIE THE POOH, STEWIE GRIFFIN, SQUIDWARD, SPONGEBOB, BUZZ LIGHTYEAR or CASPER. Mythical characters are also contemplated e.g., a cyclops, chimera, centaur, bigfoot, elf, ghost, gnome, sprite, zombie, vampire or a unicorn. Movie or cartoon characters are also contemplated e.g., DARTH VADER, THOR, HARRY POTTER, DUMBLEDORE, LOKI, TINKERBELL, DRACULA, PRINCESS MONONOKE, ARIEL or TOOTHLESS. Images of movie, TV and video game stars are also contemplated. Trading card characters and videogame characters are also contemplated.

Means to attach the mask to the patients head include, for example, string or elastic straps which can go around the patient's ears or around the head.

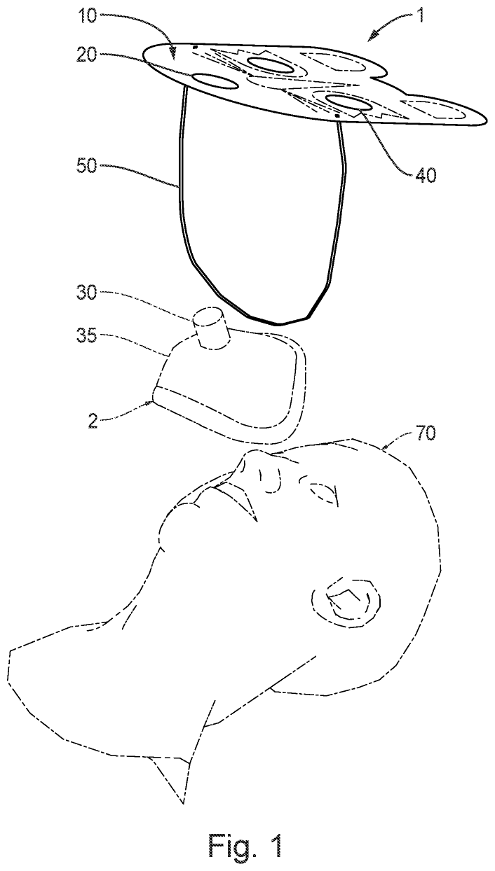

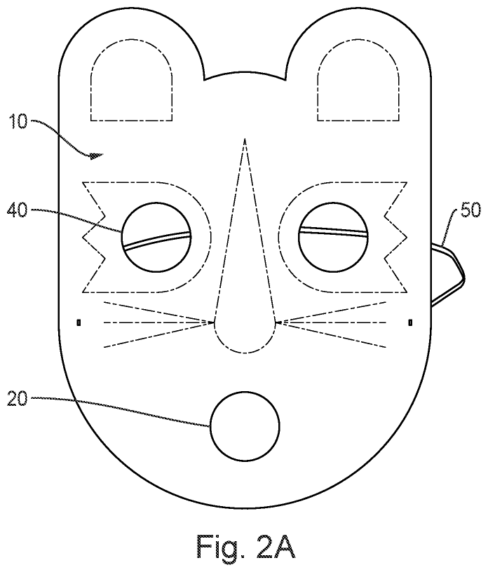

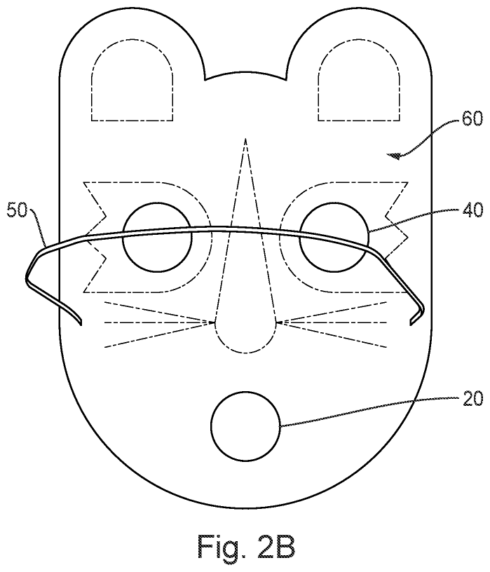

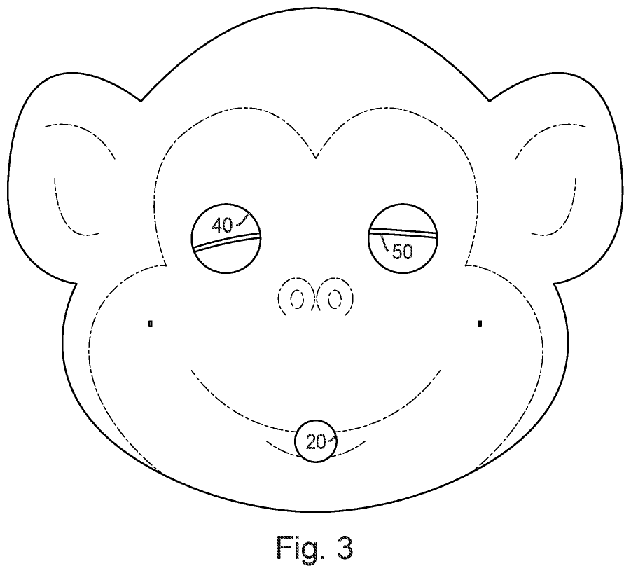

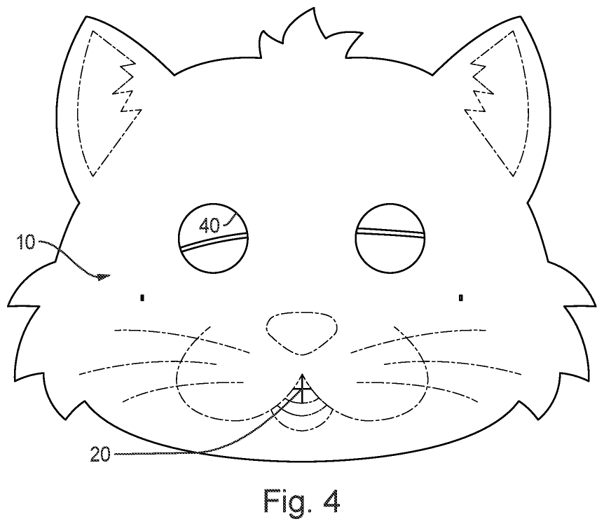

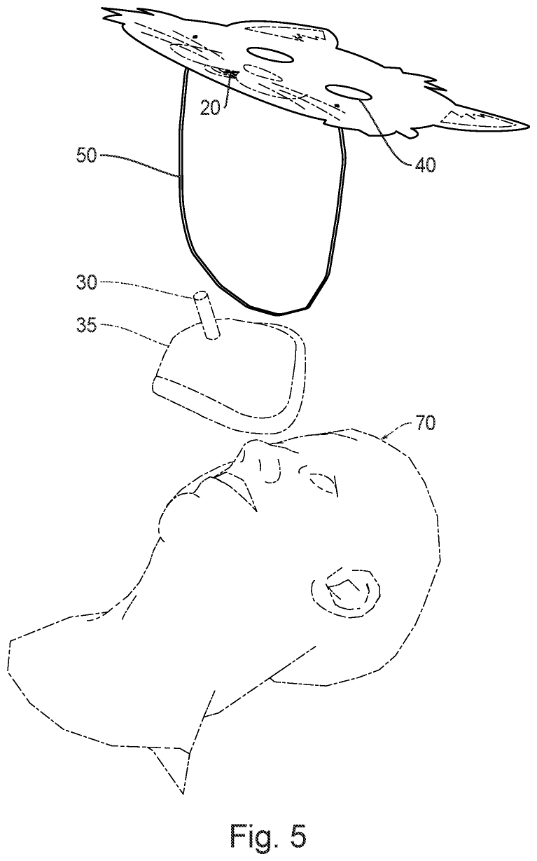

FIG. 1 depicts a layered view of a double sided facemask (1) of the present invention. The backside of the mask is not visible in this view. A typical anesthesia mask (2) has an airway device gas line opening (30). See, for example, element 126 in U.S. Pat. No. 8,336,549. In use, the anesthesia mask (2) fits over the patient's nose/mouth and the facemask aperture (20) fits over the airway device gas line opening on the anesthesia mask. Eye apertures (40) allow for viewing of the patient and depending on the placement may also allow viewing of mask fogging. FIG. 2 depicts the front side (A) and the back side (B) of a double sided facemask (1) of the present invention. In certain embodiments the front and the back surface of the double sided facemask are printed with the same face representation. In other embodiments, the front surface and the back surface are different. For example, the front may be printed with an animal face and the back surface may be printed with a cartoon character. FIG. 3 depicts the front side of a double sided facemask (1) of the present invention. In this embodiment, the facemask aperture (20) is sized to fit a thin airway tube such as shown in the airway device of FIG. 5. FIG. 4 depicts the front side of a double sided facemask (1) of the present invention. In this embodiment, the facemask aperture (20) is a slit design which can accommodate multiple size airway devices. FIG. 5 depicts a layered view of an embodiment of the present invention showing the front side of a double sided facemask (1) of the present invention in relation to a patient wearing an airway device. The backside of the mask is not visible in this view. In this embodiment, the facemask aperture (20) is a slit design which can accommodate multiple size airway devices.

There are numerous airway devices having gas line openings (30) of various diameters. Airway Mask is the term that can be used for a nose/mouth covering mask which is often used for supplemental oxygen or other gas that can be used with an Ambu bag or a ventilator, respirator etc. Such airway devices include, for example, Simple Facemasks, Nor-rebreather Masks, Nasal Cannulae, CPAP/BiPAP masks and Nebulizer masks.

The entire disclosure of all applications, patents and publications, cited herein are incorporated by reference herein.

From the foregoing description, one skilled in the art can easily ascertain the essential characteristics of this invention and, without departing from the spirit and scope thereof, can make various changes and modifications of the invention to adapt it to various usages and conditions.

We claim:

1. A face mask having:
a front surface representing a face (10),
a back surface representing a face (60)
and
a mouth or nose aperture (20)
wherein said mouth or nose aperture is adapted to couple with an airway device gas line opening (30).

2. The face mask of claim 1, wherein said mask is stiff enough to resist bending or folding under its own weight once coupled to said airway device.

3. The face mask of claim 1, wherein said face mask further contains at least one eye aperture (40).

4. The face mask of claim 1, wherein said face mask is paper, plastic or foam.

5. The face mask of claim 4, wherein said face mask is PVC plastic.

6. The face mask of claim 4, wherein said face mask is EVA flexible foam.

7. The face mask of claim 4, wherein said face mask is 200-300 GSM cardboard.

8. The face mask of claim 1, wherein said mask further comprises elastic straps (50) adapted to secure and adjust said facemask to the head of a patient (70).

9. The face mask of claim 1, wherein said mask is from 0.4 to 5 mm thick.

10. The face mask of claim 1, wherein said face mask is from about 5-9 inches wide and from about 7 to 14 inches high.

11. The face mask of claim 1, wherein said aperture adapted to couple with said airway device gas line opening represents the mouth of said face.

12. The face mask of claim 1, wherein said back surface representing a face contains at least one eye aperture and further printed indicia representing eyelash, eyebrow, skin fold, ear, nose, forehead, chin, cheek or beak.

13. A face mask having:
a front surface representing a face (10),
a back surface (61)
and
a mouth or nose aperture (20)
wherein said mouth or nose aperture is adapted to couple with an airway device gas line opening (30) and wherein said mask is flat.
